(19) United States
(12) Patent Application Publication
(10) Pub. No.: US 2021/0332001 A1
(43) Pub. Date: Oct. 28, 2021

(54) SYNTHESIS AND ANTI-TUMOR ACTIVITIES OF ACYL-PARA-AMINOPHENOL DERIVATIVES

(71) Applicant: Montefiore Medical Center, Bronx, NY (US)

(72) Inventors: Parviz LALEZARI, Bronx, NY (US); Rukmani LEKHARJ, Bronx, NY (US); Alex LUNA, Bronx, NY (US); Diana CASPER, Bronx, NY (US)

(21) Appl. No.: 17/308,507

(22) Filed: May 5, 2021

Related U.S. Application Data
(63) Continuation of application No. PCT/US19/59885, filed on Nov. 5, 2019.
(60) Provisional application No. 62/755,599, filed on Nov. 5, 2018.

(51) Int. Cl. C07C 231/02; C07C 231/24

(52) U.S. Cl. CPC C07C 231/02; C07C 231/24

(57) ABSTRACT

Method are disclosed for synthesizing derivatives of acyl-para-aminophenol and for the use of the compounds for treating lymphomas and tumors of the brain and spinal cord.

### CROSS-REFERENCE TO RELATED APPLICATION(S)

This present application is a continuation of International Patent Application No. PCT/US2019/059885 entitled “SYNTHESIS AND ANTI-TUMOR ACTIVITIES OF ACYL-PARA-AMINOPHENOL DERIVATIVES” filed on Nov. 5, 2019, which claims priority to U.S. Provisional Application No. 62/755,599, filed Nov. 5, 2018.

### BACKGROUND

Throughout this application various publications are referred to in parentheses. Full citations for these references may be found at the end of the specification. The disclosures of all publications, patents and patent applications mentioned herein are hereby incorporated by reference in their entirety into the subject application to more fully describe the art to which the subject invention pertains.

N-(4-hydroxyphenyl) acetamide (also known as acetyl-para-aminophenol, APAP, or TYLENOL®) was originally synthesized by Morse in 1878 (1), and its analgesic and anti-pyretic effects were demonstrated by Cahn and Hepp in 1886 (2). There has been a recent interest in APAP and non-steroidal anti-inflammatory drugs (NSAIDS) such as aspirin and ibuprofen, because of reports of a reduction in the incidence of various forms of cancer in patients who chronically use them (3-5). Casper et al. explored the anti-tumor activities of APAP and demonstrated its ability to reduce the growth of malignant astrocytes by cell culture method (6). These effects, however, were only mild.

Various derivatives of para-aminophenol (PAP) have been previously synthesized (7) and many have been tested for anti-tumor activities. However, the methods employed for their synthesis have been complicated, and their potential anti-tumor activities against glioblastoma multiforme (GBM) and lymphoma cells have not been explored. Over several decades numerous methods for synthesis of PAP-derivatives have been described. As an example, a recent reference for synthesis of acetyl-para-aminophenol uses hydroquinone, ammonium acetate and acetic acid mixed in Argon atmosphere, and heated at 230° C. for 15 hours. After cooling, acetic acid is evaporated, the precipitate is washed and dried (8).

### SUMMARY

Provided herein are novel methods for synthesizing and crystalizing derivatives of acyl-para-aminophenol. These methods rapidly produce high yield compounds with high purities, and may have additional applications for acylating other amino compounds. As shown herein, the resultant compounds exhibit in vitro anti-tumor toxicity effects against GBM and lymphoma cell lines. Accordingly, the present invention further provides methods of treating or preventing cancers, including GBM and lymphoma, as well as other conditions.

### BRIEF DESCRIPTION OF THE DRAWINGS

FIG. 1. U87MG cells, stage IV astrocytoma obtained from ATCC, were tested against APAP and its derivatives at various concentrations with 0.2% DMSO as a vehicle. Cells were seeded at 3500 cells and allowed 48 hours to stabilize before treatment. Drugs were administered and data for cell survival, using presto blue, were analyzed at 48 hours post treatment. 2 mM APAP demonstrated a 36% reduction in cell growth as compared to control 0.2% DMSO. The derivatives demonstrate a greater or equal activity at equal to lower concentrations. In particular PL7 and PL8 demonstrate the greatest activity at 0.5 and 0.25 mM respectively, while PL11 demonstrated a 87.5% reduction at 0.25 mM.

FIG. 2. The compounds were tested against T-cell lymphoma cells line (Jurkat cells) obtained from ATCC and were tested against APAP and its derivatives at various concentrations with 0.2% DMSO as a vehicle. Free flowing cells were seeded at 3500 cells and allowed 48 hours to stabilize before treatment. Drugs were administered to both seeded wells and wells that that were designated blank, without cells. This additional step was added in order to account for the effects of the drugs on the reagent used for analysis. Data for cell survival, using presto blue, was analyzed at 48 hours post treatment. 2 mM APAP demonstrated a 16% reduction in cell growth as compared to control 0.2% DMSO. The derivatives demonstrate greater or equal activity at equal to lower concentrations to APAP. In particular PLSV and PL7 to PL11 demonstrate the greatest activity at all concentrations used, with the exception of PL10 that demonstrated the greatest activity when compared to APAP at 0.125 mM.

FIG. 3. The compounds were tested on a differentiated neuronal cell line (9) HCN-2, obtained from ATCC. Cells were seeded at 5,000 cells per well using a low passage, second passage of cells to ensure cell stability. Cells were then subjected to induced differentiation, at 72 hours, with a differentiating concoction composed of 25 ng/mL NGF-beta-human, 0.05 mM dibutyryl cAMP and 0.5 mM IBMX in 10% DMEM, all differentiating factors were obtained from Sigma Aldrich (9). Cells were treated with differentiating factor for 12 days to ensure that cells differentiated into neuronal cells. On the twelfth day, cells were treated with and without drugs at the highest soluble concentration to determine the activity of the compounds on neuronal cells. PL7, PL8 and PL9 demonstrated toxicity at the highest concentration, 0.5 mM, 0.5 mM and 0.25 mM respectively. PL10 and PL11 demonstrated a 35.6% non-significant reduction.

### DETAILED DESCRIPTION

The present invention provides description of a new method for synthesis and crystallization of different acyl para-aminophenol derivatives. These compounds have been tested and some shown to be highly toxic against glioblastoma multiforme cell line, with less toxicity against differentiated neuronal cell line, used as control. Most of the derivatives also have been shown to be active against T-cell lymphoma cells, indicating potential effect against multiple malignancies.

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The term “treating” or “treatment” of a disease or disorder includes at least partially: (1) inhibiting the disease, disorder, or condition, i.e., arresting or reducing the development of the disease, disorder, or condition or its clinical symptoms; or (2) relieving the disease, disorder, or condition, i.e., causing regression of the disease, disorder, or condition or its clinical symptoms.

The term “preventing” or “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

### Methods of Synthesizing and Crystallizing Acyl-Para-Aminophenol Derivatives

Provided herein are methods for synthesizing acyl-para-aminophenol derivatives.

In some embodiments, the methods for synthesizing acyl-para-aminophenol derivatives comprise:

i) dissolving para-aminophenol (PAP) in a solvent to form a PAP solution;

ii) adding a base to the PAP solution to form a PAP-base solution;

iii) adding an acylating agent to the PAP-base solution to form a solution comprising base and PAP precipitates and the acyl-para-aminophenol derivatives;

iv) removing the base and PAP precipitates from the solution; and

v) retrieving the acyl-para-aminophenol derivatives from the solution.

In another embodiment, the methods for synthesizing acyl-para-aminophenol derivatives comprise:

i) dissolving PAP in tetrahydrofuran (THF) to form a PAP solution;

ii) adding triethylamine (TEA) to the PAP solution and stirring at room temperature (e.g., 23° C.) to form a PAP-TEA solution;

iii) adding an acylating agent to the PAP-TEA solution to form TEA and PAP precipitates and the acyl-para-aminophenol derivatives;

iv) removing TEA and PAP precipitates by filtration to obtain a THF filtrate; and

v) retrieving the acyl-para-aminophenol derivatives from the THF filtrate.

In some embodiments, the methods for synthesizing acyl-para-aminophenol derivatives comprise:

i) dissolving PAP in THF to form a PAP solution at room temperature;

ii) stirring the PAP solution at room temperature;

iii) adding TEA to the stirred PAP solution to form a PAP-TEA solution;

iv) stirring the PAP-TEA solution at room temperature;

v) adding an acylating agent to the stirred TEA-PAP solution;

vi) stirring the solution to form TEA-HCl and PAP-HCl precipitates and the acyl-para-aminophenol derivatives;

vii) removing the TEA-HCl and PAP-HCl precipitates by filtration to obtain a THF filtrate; and

viii) retrieving the acyl-para-aminophenol derivatives from the THF filtrate.

In some embodiments, the base is an organic base. In some embodiments, the organic base is an amine. In some embodiments, the amine is a tertiary amine. Non-limiting examples of tertiary amines include triethylamine (TEA) and diisopropylethylamine (DIPEA).

In some embodiments, the solvent is an organic solvent. In some embodiments, the organic solvent is any solvent that can dissolve PAP. In some embodiments, the organic solvent is an ether. For example, in some embodiments, the solvent is THF, diethyl ether, or 1,4-dioxane.

In some embodiments, the methods comprise dissolving PAP in about 60 to about 80 mL of the solvent. For example, about 60 mL, about 65 mL, about 70 mL, about 75 mL, or about 80 mL of the solvent. In some embodiments, the solvent is an organic solvent. In some embodiments, the solvent is THF.

In some embodiments, the methods comprise stirring the PAP and the solvent at room temperature for about 5 minutes to about 10 minutes. For example, about 5 minutes, about 6 minutes, about 7 minutes, about 8 minutes, about 9 minutes, or about 10 minutes. In some embodiments, the solvent is an organic solvent. In some embodiments, the solvent is THF.

In some embodiments, the methods comprise adding about 2 mM to about 3 mM of base to the PAP solution. For example, about 2 mM, about 2.1 mM, about 2.2 mM, about 2.3 mM, about 2.4 mM, about 2.5 mM, about 2.6 mM, about 2.7 mM, about 2.8 mM, about 2.9 mM, or about 3 mM of the base. In some embodiments, the base is TEA. In some embodiments, the PAP solution is PAP dissolved in an organic solvent. In some embodiments, the organic solvent is THF.

In some embodiments, the methods comprise stirring the base and the PAP solution at room temperature for about 2 minutes to about 5 minutes. For example, about 2 minutes, about 2.5 minutes, about 3 minutes, about 3.5 minutes, about 4 minutes, about 4.5 minutes, or about 5 minutes. In some embodiments, the PAP solution is PAP dissolved in an organic solvent. In some embodiments, the base is TEA.

In some embodiments, the methods comprise adding 4 mM to about 10 mM of an acylating agent to the PAP-base solution. For example, about 4 mM, about 6 mM, about 8 mM, or about 10 mM of an acylating agent. In some embodiments, the PAP-base solution comprises an organic solvent. In some embodiments, the organic solvent is THF. In some embodiments, the base is TEA.

In some embodiments, the methods comprise adding 4 mM to about 10 mM of PAP to the solvent. For example, about 4 mM, about 6 mM, about 8 mM, or about 10 mM of PAP. In some embodiments, the organic solvent is THF. In some embodiments, about 4 mM of PAP is dissolved in about 60 mL to about 80 mL of the organic solvent. In some embodiments, the organic solvent is THF.

In some embodiments, the methods comprise stirring the acylating agent in the PAP-base solution for about 20 minutes to about 40 minutes. For example, about 20 minutes, about 25 minutes, about 30 minutes, about 35 minutes, or about 40 minutes. In some embodiments, the methods comprise stirring at room temperature (e.g., 23° C.) or at about 40° C. to about 70° C. For example, at about 40° C., about 45° C., about 50° C., about 55° C., about 60° C., about 65° C., or about 70° C. In some embodiments, the base is TEA.

In some embodiments, when the acylating agent comprises 6-16 carbons, the acylating agent is added to the PAP-base solution at about 60° C. In another embodiment, when the acylating agent is an acetyl, propionyl, butyric, or valéry structure, the acylating agent is added room temperature to the base-PAP solution. In some embodiments, the base is TEA.

In some embodiments, the methods for synthesizing acyl-para-aminophenol derivatives comprise:

i) dissolving about 4 mM of PAP in about 60 to about 80 mL of THF to form a PAP solution;

ii) stirring the PAP solution at room temperature for about 5 minutes to about 10 minutes;

iii) adding about 2 mM to about 3 mM, preferably about 2.5 mM, of TEA to the PAP solution to form a PAP-TEA solution;

iv) stirring the PAP-TEA solution at room temperature for about 2 minutes to about 5 minutes;

v) adding 4 mM of an acylating agent to the PAP-TEA solution;

vi) stirring the PAP-TEA solution for about 20 to about 40 minutes, preferably about 30 minutes, at room temperature or at about 40° C. to about 70° C., preferably at about 60° C., to form a precipitate comprising TEA-HCl and PAP-HCl;

vii) removing the precipitate comprising TEA-HCl and PAP-HCl by filtration to obtain a THF filtrate; and

viii) retrieving the acyl-para-aminophenol derivatives from the THF filtrate.

The acylating agent can be, for example, acyl fluoride, acyl chloride or acyl bromide. The acylating agent can be, for example, selected from the group consisting of acetyl, propionyl, butyryl, isobutyryl, valeryl, hexanoyl, heptanoyl, octanoyl, nonanoyl, decanoyl, dodecanoyl, miristyl, benzoyl, naphthoyl, hexadecanoyl and oleoyl chlorides. In one embodiment, the acylating agent is an acetyl, propionyl, butyric or valéry structure, and step vi) is carried out at room temperature. In another embodiment, the acylating agent comprises 6-16 carbon atoms, and step vi) is carried out at about 60° C.

The method can further comprise crystallization of the acyl-para-aminophenol derivative by suspending the derivative in distilled water, then solubilizing the derivative by adding an organic solvent (e.g., ethanol) to generate crystals by gradual cooling; and separating crystals from the organic solvent-water mixture by filtration and drying. The amounts of distilled water can, for example, range from 10 to 100 ml. In some embodiments, the distilled water can be in an amount of about 10 to ml, about 20 ml, about 30 ml, about 40 ml, about 50 ml, about 60 ml, about 70 ml, about 80 ml, about 90 ml, or about 100 ml. The amounts of the organic solvent can be, for example, from zero to 150 ml, depending on the structure of the derivative. In some embodiments, no organic solvent (i.e., zero) is added to crystallize the acyl-para-aminophenol derivatives. In another embodiment, organic solvent (e.g., ethanol) is added in about 10 ml, about 20 ml, about 30 ml, about 40 ml, about 50 ml, about 60 ml, about 70 ml, about 80 ml, about 90 ml, about 100 ml, about 110 ml, about 120 ml, about 130 ml, about 140 ml, or about 150 ml. Variable degrees of heat, for example, from room temperature to 100° C. can be used to dissolve the acyl-para-aminophenol derivative, depending on the structure of the acyl-para-aminophenol derivative. In some embodiments, the derivative is dissolved at a temperature of about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C.

In some embodiments, the crystallization of the acyl-para-aminophenol derivatives is achieved using an organic solvent that can solubilize the acyl-para-aminophenol derivatives. In some embodiments, the organic solvent is an alcohol. For example, in some embodiments, the derivative is crystallized from ethanol, isopropanol, methanol, or other alcohol-based solvent. In some embodiments, the organic solvent is not an alcohol-based solvent.

In some embodiments, the acyl-para-aminophenol derivatives are crystallized using evaporation techniques, cooling techniques (e.g., dissolving the compound in an organic solvent and reducing the temperature to about 4° C. or less), dissolving the acyl-para-aminophenol derivatives in a first solvent and then adding a second solvent to reduce the solubility of the acyl-para-aminophenol derivatives, by layering different solvents (e.g., layering different solvents selected based on the solubility of the acyl-para-aminophenol derivatives), or sublimation.

In some embodiments, the acyl-para-aminophenol derivative is a solid when retrieved from the solution (e.g., filtrate), wherein the solution comprising an organic solvent (e.g., THF). In some embodiments, the acyl-para-aminophenol derivative is a white, tan, off-white, yellow, pale pink, beige, or grey solid. In some embodiments, the solid acyl-para-aminophenol derivative is removed from the solution of organic solvent by evaporation and/or drying. In some embodiments, the organic solvent is removed by placing the solid acyl-para-aminophenol derivative in the organic solvent under reduced pressure (e.g., rotary evaporation or vacuum). In some embodiments, the organic solvent is removed by distillation. In some embodiments, the organic solvent is removed by gravity or vacuum filtration. In some embodiments, the solvent is removed from the solid acyl-para-aminophenol derivative by an open-dish evaporation (e.g., “air” drying).

In some embodiments, the solid acyl-para-aminophenol derivative can be retrieved from the THF filtrate by evaporation of THF and/or drying of the THF. In some embodiments, the solid acyl-para-aminophenol derivate can be retrieved from the THF filtrate by removing the THF under reduced pressure. In one embodiment, the THF can be removed by distillation. In another embodiment, the solid acyl-para-aminophenol derivative can be retrieved from the THF filtrate by gravity or vacuum filtration. In some embodiments, the THF is removed by open-dish evaporation.

In some embodiments, the derivative of acyl-para-aminophenol derivatives are compounds of formula (I) having the structure:

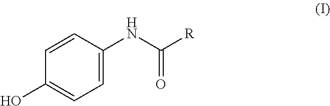

wherein R is a C2-C15 straight chain or branched alkyl, alkenyl, or alkynyl, or a cycloalkyl, heterocycloalky, aryl, heteroaryl, aralkyl, or heteroaralkyl.

The R group can be optionally substituted with, for example, one or more of F, Cl, Br, I, OH, SH, and NO₂. In one embodiment, R is a straight chain C6-C8 alkyl.

In some embodiments, the acyl-para-aminophenol derivatives can be, for example, acetyl-para-aminophenol, N-(4-hydroxyphenyl)propanamide, N-(4-hydroxyphenyl)-2-methylpropanamide, 4′-Hydroxybutyranilide, N-(4-hydroxyphenyl)pentanamide, N-(4-hydroxyphenyl)benzamide, N-(4-hydroxyphenyl)hexanamide, N-(4-hydroxyphenyl)heptanamide, N-(4-hydroxyphenyl)octanamide, N-(4-hydroxyphenyl)nonanamide, N-(4-hydroxyphenyl)decanamide, N-(4-hydroxyphenyl)-1-naphthamide, N-(4-hydroxyphenyl)-2-naphthamide, N-(4-hydroxyphenyl)dodecanamide, N-(4-hydroxyphenyl)tetradecanamide, or N-(4-Hydroxyphenyl)hexadecanamide.

In some embodiments, the methods provided herein include synthesizing and crystallizing acyl para-aminophenol derivatives on a large, industrial scale (e.g., mass production). For mass production, the acyl-para-aminophenol derivatives are synthesized by using equimolar concentrations of para-aminophenol and the desired acylating agent ranging each from about 1 mM to about 10 M. For example, the concentrations PAP and acylating agent used are about 1 mM, about 10 mM, about 100 mM, about 1 M, or about 10 M. For the mass production of the acyl para-aminophenol derivatives, the organic solvent (e.g., THF) and base (e.g., TEA) for the synthesis phase and distilled water and organic solvent (e.g., ethanol) for crystallization are used in volumes proportional in the ranges and amounts describe for the general syntheses above.

In some embodiments, the methods for the mass production of acyl para-aminophenol derivatives can generate about 10 g to about 1,000 g, or more of the acyl para-aminophenol derivatives. For example, about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, about 360 g, about 370 g, about 380 g, about 390 g, about 400 g, about 410 g, about 420 g, about 430 g, about 440 g, about 450 g, about 460 g, about 470 g, about 480 g, about 490 g, about 500 g, about 600 g, about 700 g, about 800 g, about 900 g, about 1,000 g, or more.

### Methods of Treating or Preventing

Also provided herein are methods of using acyl-para-aminophenol derivatives to treat or prevent various conditions.

In certain embodiments, methods are provided for treating or preventing a tumor of the brain or spinal cord or a lymphoma in a subject comprising administering to the subject a compound of formula (I) in an amount and manner effective to inhibit the growth of the tumor cells, wherein the compound of formula (I) has the structure:

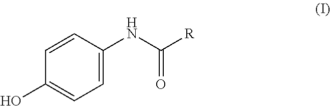

wherein R is a C2-C15 straight chain or branched alkyl, alkenyl, or alkynyl, or a cycloalkyl, heterocycloalky, aryl, heteroaryl, aralkyl, or heteroaralkyl.

In certain embodiments, methods are provided for enhancing the radiosensitivity of a tumor of the brain or spinal cord or of a lymphoma in a subject comprising administering to the subject a compound of formula (I) in an amount and manner effective to enhance the radiosensitivity of the tumor, wherein the compound of formula (I) has the structure:

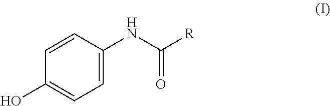

wherein R is a C2-C15 straight chain or branched alkyl, alkenyl, or alkynyl, or a cycloalkyl, heterocycloalky, aryl, heteroaryl, aralkyl, or heteroaralkyl.

The R group can be optionally substituted with, for example, one or more of F, Cl, Br, I, OH, SH, and NO₂. In one embodiment, R is straight chain C6-C8 alkyl.

In certain embodiments of the methods provided herein, the acyl-para-aminophenol derivatives may be selected from, for example, N-(4-hydroxyphenyl)-2-methylpropanamide, 4′-Hydroxybutyranilide, N-(4-hydroxyphenyl)pentanamide, N-(4-hydroxyphenyl)benzamide, N-(4-hydroxyphenyl)hexanamide, N-(4-hydroxyphenyl)heptanamide, N-(4-hydroxyphenyl)octanamide, N-(4-hydroxyphenyl)nonanamide, N-(4-hydroxyphenyl)decanamide, N-(4-hydroxyphenyl)-1-naphthamide, N-(4-hydroxyphenyl)-2-naphthamide, N-(4-hydroxyphenyl)dodecanamide, N-(4-hydroxyphenyl)tetradecanamide, or N-(4-Hydroxyphenyl)hexadecanamide.

Also provided herein are methods of treating various forms of malignancies by combining the described derivatives with other known chemotherapeutic agents or radiation and enhance their efficacy. For example, in some embodiments, the malignancies include carcinoma, sarcoma, melanoma, lymphoma, and leukemia. In some embodiments, the malignancy is associated with a specific organ such as the skin, lungs, breasts, brain, or pancreas.

The tumor can be, for example, a glioblastoma.

In some embodiments, enhancing radiosensitivity includes increasing the relative responsiveness of cells, tissues, and/or organs to radiation treatment.

In some embodiments, the subject has symptoms of glioblastoma. Symptoms of glioblastoma include, but are not limited to, headaches, nausea, decline in brain function, memory loss, personality changes, difficulty balancing, urinary incontinence, vision impairments, speech difficulties, and/or seizures.

In some embodiments the subject has a risk factor for cancer (e.g., brain or spinal cord tumor or lymphoma). Non-limiting examples of risks factors include age (e.g., between 45 to 65 years old), exposure to radiation, or a family history of cancer.

In some embodiments, the subject has a genetic predisposition for glioblastoma. In some embodiments, the subject does not have a genetic predisposition for glioblastoma.

In some embodiments, the subject has a glioma selected from the group consisting of astrocytomas, ependymomas, and oligodendrogliomas. In some embodiments, the astrocytomas includes astrocytoma, anaplastic astrocytoma, and glioblastoma. In some embodiments, the ependymomas includes anaplastic ependymoma, myxopapillary ependymoma, and subependymoma. In some embodiments, the oligodendrogliomas include oligodendroglioma, anaplastic oligodendroglioma, and anaplastic oligoastrocytoma.

All combinations of the various elements described herein are within the scope of the invention unless otherwise indicated herein or otherwise clearly contradicted by context.

Where a numerical range is provided herein, it is understood that all numerical subsets of that range, and all the individual integers contained therein, are provided as part of the invention.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Overview

This study describes a simple and rapid method for synthesis of several para aminophenol (PAP) derivatives, which were found to be more effective against glioblastoma multiforme cell lines and T-cell lymphoma (Jurkat) cells with lower toxicity against neuronal cells, demonstrating promising effects against brain tumor and lymphoma cell lines.

### Materials and Methods

General Synthesis Method: For synthesis of each compound 4 mM of PAP were dissolved in 60-80 mL of Tetrahydrofuran (THF) and after five minutes stirring at room temperature 2.5 mM of Triethylamine (TEA) were added and after 2-5 minutes stirring at room temperature, 4 mM acylating agent were added. The acylating agents used included acetyl, propionyl, butyryl, isobutyryl, valeryl, hexanoyl, heptanoyl, octanoyl, nonanoyl, decanoyl, dodecanoyl, miristyl, benzoyl, naphthoyl, and hexadecanoyl chlorides. The reaction was continued either at room temperature or at 60° C. based on the structure of the acylating agent for 30 minutes. Although all compounds could be synthesized at either temperature, the best results could be obtained at room temperature for acetyl, propionyl, butyryl and Valéry structures. 60° C. provided better results when acylating agents with higher number of carbons were used (from 6 to 16). The use of high temperature required the use of a ferflux device. During stirring at the desired temperatures, a white precipitate was rapidly developed that was composed of TEA-HCl and small amount of PAP-HCl. The precipitate was removed by filtration. The acylated derivatives that are soluble in THF, were retrieved in the THF filtrate. Solid acylated compounds were then obtained after cooling and evaporation of THF, and drying.

For crystallization, the general method was to disperse the products in distilled water (DW) and then add ethanol to dissolve the compounds and generate crystals by gradual cooling. The final crystals then were separated by filtration and dried. The crystallization method varied for compounds with different number of carbons in the acylating agents. For crystallization of APAP, ethanol could not be used, because the high solubility of APAP in ethanol prevents its crystallization. For all other compounds, crystallization required a combination of ethanol and DW, lower proportion of ethanol being needed for compounds with lower number of carbons. For example, for crystallization of propionyl-PAP, the dried product obtained after THF evaporation required 30 mL of DW and only 7 ml of ethanol, whereas for larger molecules such as hexanoyl-PAP, 60 ml of DW and 15 ml of ethanol were required. Moderate heat was helpful to dissolve all compounds, and for compounds with larger carbon numbers higher temperatures and larger ethanol volumes were needed.

Testing of Compounds: All the compounds have been evaluated experimentally for anti-tumor activities against SNB-19 astrocytoma, U87-MG glioblastoma, neuronal cells and Jurkat (T-cells) lymphoma cell lines.

### Results

The molecular structures, molecular weights (MW) determined by Mass spectroscopy, and melting points (MP) of the compounds synthesized are shown in Table 1. All the compounds were analyzed by NMR and Mass spectroscopy and the results confirmed their structures as expected.

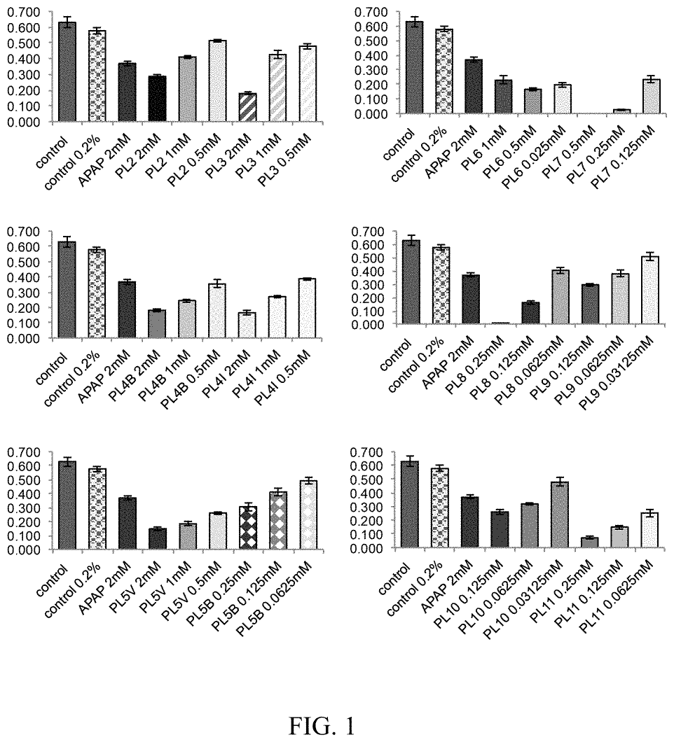

U87MG cells, stage IV astrocytoma obtained from ATCC, were tested against APAP and its derivatives at various concentration with 0.2% DMSO as a vehicle (FIG. 1). Cells were seeded at 3500 cells and allowed 48 hours to stabilize before treatment. Drugs were administered and data for cell survival, using presto blue, were analyzed at 48 hours post treatment. 2 mM APAP demonstrated a 36% reduction in cell growth as compared to control 0.2% DMSO. The derivatives demonstrate a greater or equal activity at equal to or lower concentrations. In particular PL7 and PL8 demonstrate the greatest activity at 0.5 and 0.25 mM respectively, while PL11 demonstrated a 87.5% reduction at 0.25 mM.

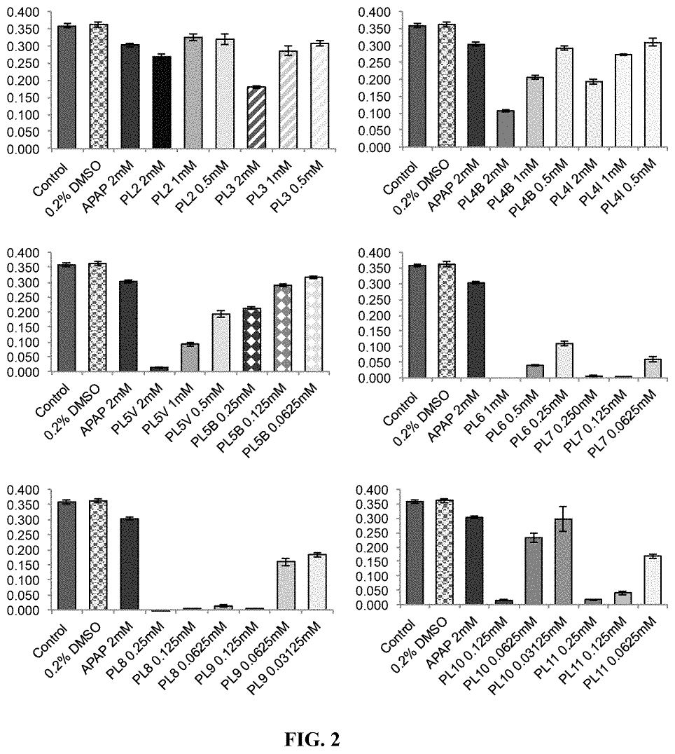

The effects of the drugs on lymphoma cells were tested on Jurkat cells, as described above and in FIG. 2.

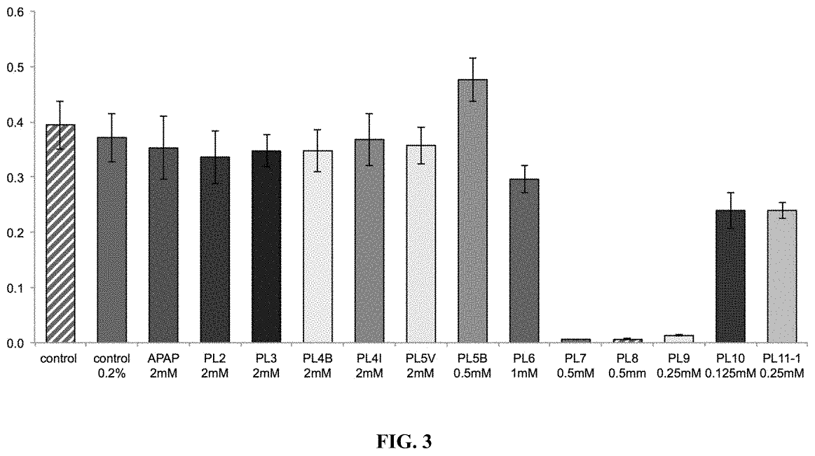

The compounds were also tested on a differentiated neuronal cell line, HCN-2, obtained from ATCC (FIG. 3). PL7, PL8 and PL9 demonstrated toxicity at the highest concentration, 0.5 mM, 0.5 mM and 0.25 mM respectively. PL11 and P110 demonstrated a 35.6% reduction, however it was not a significant reduction.

### DISCUSSION

Previously described synthesis methods require long procedures and equipment that are not found in many labs. The new procedure can be done in any lab with a fume hood, heater and a reflux device with maximum synthesis time of one hour, even at room temperature with a minor reduction of yield. The high in vitro activities of the compounds against GBM and Jurkat cell lines indicates potential therapeutic effects for glioblastoma, which currently is resistant to other treatments, lymphomas and many other malignancies. The compounds may also increase radiosensitivity of solid tumors.

TABLE 1
Structure, MW (confirmed by Mas Spec) and MP of the
compounds synthesized.

| | | | | | Predicted | | |
|---|---|---|---|---|---|---|---|
| | | | | | m/z | | Error |
| | STRUCTURE | FORMULA | MW | MP ° C. | [M + H]⁺¹ | Observed | (ppm) |
| PL2 | 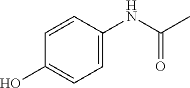 | C₈H₉NO₂ | 151.2 | 169 | 152.0706 | 152.0706 | 0 |
| PL3 | 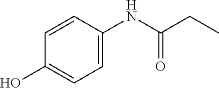 | C₉H₁₁NO₂ | 165.192 | 171 | 166.0863 | 166.0862 | −0.6 |
| PL4 | 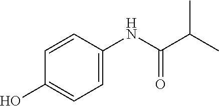 | C₁₀H₁₃NO₂ | 179.219 | 160-162 | 180.1019 | 180.1019 | 0 |
| PL5V | 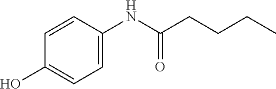 | C₁₁H₁₅NO₂ | 193.246 | 96 | 194.1176 | 194.1174 | −1.0 |
| PL5B | 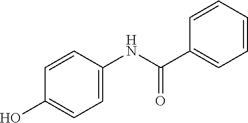 [SMILES: O=C(Nc1ccc(O)cc1)c1ccccc1] | C₁₃H₁₁NO₂ | 216.079 | 217-218 | 214.0863 | 214.0861 | 0.9 |
| PL6 | 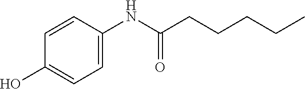 | C₁₂H₁₇NO₂ | 207.273 | 110 | 208.1332 | 208.1331 | −0.5 |
| PL7 | 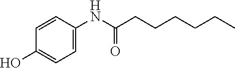 [SMILES: CCCCCCC(=O)Nc1ccc(O)cc1] | C₁₃H₁₉NO₂ | 221.3 | 109 | 222.1489 | 222.1487 | −0.9 |
| PL8 | 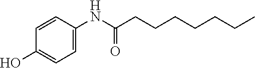 [SMILES: CCCCCCCC(=O)Nc1ccc(O)cc1] | C₁₄H₂₁NO₂ | 235.327 | 120 | 236.1645 | 236.1644 | −0.4 |
| PL9 | 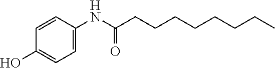 [SMILES: CCCCCCCCC(=O)Nc1ccc(O)cc1] | C₁₅H₂₃NO₂ | 249.354 | 119 | 250.1802 | 250.1800 | −0.8 |
| PL10 | 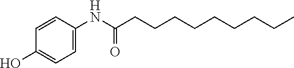 [SMILES: CCCCCCCCCC(=O)Nc1ccc(O)cc1] | C₁₆H₂₅NO₂ | 263.381 | 124 | 264.1958 | 264.1957 | −0.4 |
| PL11 | 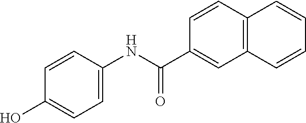 [SMILES: O=C(Nc1ccc(O)cc1)c1ccc2ccccc2c1] | C₁₇H₁₃NO₂ | 263.296 | 192-193 | 264.1019 | 264.1017 | −0.8 |
| PL12 | 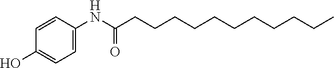 [SMILES: CCCCCCCCCCCC(=O)Nc1ccc(O)cc1] | C₁₈H₂₉NO₂ | 291.435 | 128 | 292.2271 | 292.2270 | −0.3 |
| PL14 | 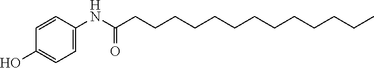 [SMILES: CCCCCCCCCCCCCC(=O)Nc1ccc(O)cc1] | C₂₀H₃₃NO₂ | 319.5 | 130 | 320.2584 | 320.2584 | 0 |
| PL16 | 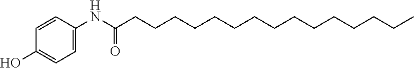 [SMILES: CCCCCCCCCCCCCCCC(=O)Nc1ccc(O)cc1] | C₂₂H₃₇NO₂ | 347.535 | 131 | 348.2897 | 348.2896 | −0.3 |
| APAP | 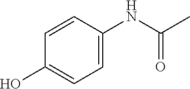 | C₈H₉NO₂ | 151.2 | 169 | 152.0706 | 152.0707 | 0.7 |

IUPAC names:
PL2: 4′-Hydroxybutyranilide
PL3: N-(4-hydroxyphenyl)propanamide
PL4: N-(4-hydroxyphenyl)-2-methylpropanamide
PL5V: N-(4-hydroxyphenyl)pentanamide
PL5B: N-(4-hydroxyphenyl)benzamide
PL6: N-(4-hydroxyphenyl)hexanamide
PL7: N-(4-hydroxyphenyl)heptanamide
PL8: N-(4-hydroxyphenyl)octanamide or (4-Caprylamidophenol)
PL9: N-(4-hydroxyphenyl)nonanamide
PL10: N-(4-hydroxyphenyl)decanamide
PL11-1: N-(4-hydroxyphenyl)-1-naphthamide or P11-2: N-(4-hydroxyphenyl)-2-naphthamide
PL12: N-(4-hydroxyphenyl)dodecanamide
PL14: N-(4-hydroxyphenyl)tetradecanamide
PL16: N-(4-Hydroxyphenyl)hexadecanamide
APAP: N-(4-Hydroxyphenyl)acetamide

### REFERENCES

1. Morse, H. N. (1878). “Ueber eine neue Darstellungsmethode der Acetylamidophenole” [On a new method of preparing acetylamidophenol]. Berichte der deutschen chemischen Gesellschaft. 11 (1): 232-3.
2. Cahn, A; Hepp P (1886). “Das Antifebrin, ein neues Fiebermittel”. Centralbl. Klin. Med. 7: 561-64.
3. Thun, M. Namboodiri, M. Aspirin use and reduced risk of fatal colon cancer. N Eng J Med 325:1593-1596, 1991.
4. Logan, R F A. Little, J. Hawtin, P G. Hardcasle, J D. Effect of aspirin and non-steroidal anti-inflammatory drugs on colorectal adenomas: case control study on subjects participating in the Nottingham faecal occult blood screening programme. Br Med J 307: 285-289, 1993.
5. Peleg, I I. Maibac, H T. Brown, S H. Wilcox, C M. Aspirin and non-steroidal anti-inflammatory drug use and the risk of subsequent colorectal cancer. Arch Intern Med 154:394-399, 1994.
6. Casper, D et al. Acetaminophen selectively reduces glioma cell growth and increases radiosensitivity in culture. J. Neuroonc (2000) 46:215-229.
7. Pubchem references: pubchem.ncbi.nlm.nih.govisearchNcollection=compounds. All compounds can be found on Pubchem using the IUPAC names.
8. Joncour, R et al. Amidation of phenol derivatives: a direct synthesis of paracetamol (acetaminophen) from hydroquinone. Green. Chem. 16: 2997-3002, 2014.
9. Zhang, Zhiyuan, et al. Human cortical neuronal (HCN) cell lines: a model for amyloid 13 neurotoxicity. Neuroscience Letters 177.1 (1994): 162-164.

1. A method of synthesizing an acyl-para-aminophenol derivative, the method comprising:
i) dissolving para-aminophenol (PAP) in a solvent to form a PAP solution;
ii) adding a base to the PAP solution to form a PAP-base solution;
iii) adding an acylating agent to the PAP-base solution to form a solution comprising base and PAP precipitates and the acyl-para-aminophenol derivative;
iv) removing the base and PAP precipitates from the solution; and
v) retrieving the acyl-para-aminophenol derivatives from the solution.

2. The method of claim 1, further comprising crystallizing the acyl-para-aminophenol derivatives.

3. The method of claim 2, wherein the crystallizing comprises suspending the acyl-para-aminophenol derivative in water, then solubilizing the acyl-para-aminophenol derivative by adding an organic solvent to generate crystals of the acyl-para-aminophenol derivative.

4. The method of claim 3, wherein the crystals of the acyl-para-aminophenol derivative are formed by gradual cooling.

5. The method of claim 3, further comprising separating the crystals from the organic solvent and water.

6. The method of claim 3, wherein the organic solvent is an alcohol-based solvent.

7. The method of claim 6, wherein the alcohol-based solvent is selected from the group consisting of ethanol, methanol, and isopropanol.

8. The method of claim 1, wherein the base is an amine.

9. The method of claim 8, wherein the amine is a tertiary amine.

10. The method of claim 9, wherein the tertiary amine is triethylamine (TEA) or diisopropylethylamine (DIPEA).

11. The method of claim 1, wherein the solvent of step i) is an organic solvent.

12. The method of claim 11, wherein the organic solvent is an ether.

13. The method of claim 12, wherein the ether is selected from the group consisting of tetrahydrofuran (THF), diethyl ether, and 1,4-dioxane.

14. The method of claim 1, wherein the PAP is dissolved in about 60 ml to about 80 ml of solvent.

15. The method of claim 1, further comprising stirring the PAP and the solvent at room temperature for about 5 minutes to about 10 minutes.

16. The method of claim 1, wherein about 4 mM to about 10 mM of PAP is dissolved in the solvent.

17. The method of claim 1, wherein about 2 mM to about 3 mM of base is added to the PAP solution.

18. The method of claim 1, further comprising stirring the PAP solution and TEA at room temperature for about 2 minutes to about 5 minutes.

19. The method of claim 1, wherein about 4 mM to about 10 mM of the acylating agent is added to the PAP-base solution.

20. The method of claim 1, further comprising stirring the acylating agent in the PAP-base solution for about 20 minutes to about 40 minutes.

21. The method of claim 1, wherein the acylating agent and PAP-base solution are stirred at room temperature or at about 40° C. to about 70° C.

22. The method of claim 1, wherein the acyl-para-aminophenol derivative is a solid when retrieved from the solution.

23. The method of claim 1, wherein the acylating agent comprises 6-16 carbons and step iii) is carried out at about 60° C.

24. The method of claim 1, wherein the acylating agent is an acetyl, propionyl, butyric, or valéry structure, and step iii) is carried out at room temperature.

25. The method of claim 1, wherein the base is TEA and the base and PAP precipitates are TEA-HCl and PAP-HCl salt precipitates.

26-33. (canceled)

34. The method of claim 1, wherein the acylating agent is selected from the group consisting of acetyl, propionyl, butyryl, isobutyryl, valeryl, hexanoyl, heptanoyl, octanoyl, nonanoyl, decanoyl, dodecanoyl, miristyl, benzoyl, naphthoyl, hexadecanoyl and oleoyl chlorides.

35. The method of claim 1, wherein the acyl-para-aminophenol derivative of acyl-para-aminophenol is selected from the group consisting of acetyl-para-aminophenol, N-(4-hydroxyphenyl)propanamide, N-(4-hydroxyphenyl)-2-methylpropanamide, 4′-Hydroxybutyranilide, N-(4-hydroxyphenyl)pentanamide, N-(4-hydroxyphenyl)benzamide, N-(4-hydroxyphenyl)hexanamide, N-(4-hydroxyphenyl)heptanamide, N-(4-hydroxyphenyl)octanamide, N-(4-hydroxyphenyl)nonanamide, N-(4-hydroxyphenyl)decanamide, N-(4-hydroxyphenyl)-1-naphthamide, N-(4-hydroxyphenyl)-2-naphthamide, N-(4-hydroxyphenyl)dodecanamide, N-(4-hydroxyphenyl)tetradecanamide or N-(4-Hydroxyphenyl)hexadecanamide.

36. (canceled)

37. A method of treating a brain or spinal cord tumor or a lymphoma in a subject comprising administering to the subject a compound of formula (I) in an amount and manner effective to inhibit the growth of the tumor cells, wherein the compound of formula (I) has the structure: 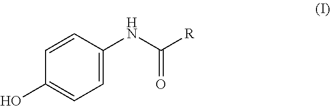
wherein R is a C2-C15 straight chain or branched alkyl, alkenyl, or alkynyl, or a cycloalkyl, heterocycloalky, aryl, heteroaryl, aralkyl, or heteroaralkyl.

38. The method of claim 37, wherein R is a straight chain C6-C8 alkyl.

39. The method of claim 37, wherein the compound is N-(4-hydroxyphenyl)-2-methylpropanamide, 4′-Hydroxybutyranilide, N-(4-hydroxyphenyl)pentanamide, N-(4-hydroxyphenyl)benzamide, N-(4-hydroxyphenyl)hexanamide, N-(4-hydroxyphenyl)heptanamide, N-(4-hydroxyphenyl)octanamide, N-(4-hydroxyphenyl)nonanamide, N-(4-hydroxyphenyl)decanamide, N-(4-hydroxyphenyl)-1-naphthamide, N-(4-hydroxyphenyl)-2-naphthamide, N-(4-hydroxyphenyl)dodecanamide, N-(4-hydroxyphenyl)tetradecanamide or N-(4-Hydroxyphenyl)hexadecanamide.

40. The method of claim 37, wherein the tumor is a glioblastoma multiforme.

41-44. (canceled)

45. The method of claim 37, wherein the compound is used in combination with radiotherapy and/or one or more other chemotherapeutic agents.

46-47. (canceled)
